(19) United States
(12) Patent Application Publication
(10) Pub. No.: US 2017/0047059 A1
(43) Pub. Date: Feb. 16, 2017

(54) PORTABLE HEARING TEST APPARATUS

(71) Applicant: HIT INCORPORATED, New Taipei City (TW)

(72) Inventors: Sung-Shou YANG, New Taipei City (TW); Shuo-Hung CHU, New Taipei City (TW)

(21) Appl. No.: 14/823,252

(22) Filed: Aug. 11, 2015

(51) Int. Cl. G10K 11/178; A61B 5/00; A61B 5/12

(52) U.S. Cl. CPC G10K 11/1782; A61B 5/123; A61B 5/6898; G10K 2210/3044; G10K 2210/3011

(57) ABSTRACT

A portable hearing test apparatus is arranged in an earphone and includes a microphone, an automatic noise elimination module, a mixer and an output unit. The microphone receives an external noise signal. The automatic noise elimination module converts the external noise signal into an inverse acoustic signal and sends the inverse acoustic signal to the mixer to mix with an audio signal provided by an electronic apparatus to obtain a mixed signal. Amplitude of the inverse acoustic signal is equal to amplitude of the external noise signal. Phase of the inverse acoustic signal and phase of the external noise signal are reverse. The mixer sends the mixed signal to the output unit to send out to cancel out the external noise signal, so that only the audio signal provided by the electronic apparatus is heard. A hearing test is proceeded with the audio signal.

### BACKGROUND OF THE INVENTION

Field of the Invention

The present invention relates to a hearing test apparatus, and especially relates to a portable hearing test apparatus for self-test.

Description of the Related Art

Sound wave is intangible energy in the air. Sound wave is collected by auricles, and is transmitted to the brain through the conchae, the middle ears and the inner ears. The brain interprets the sound wave as useful messages. Therefore, when any one of the conchae, the middle ears, the acoustic nerves in the inner ears and brain hearing center is sick, the hearing disorder happens. When the hearing disorder happens, the patient may request other people to repeat the words that are just said, especially in the noisy environment. The patient may volume up the voice of the radio or the television in the house, and may feel that the voice of other people is weak. As hearing continuously deteriorates, the patient has to pay more energy to communicate with other people.

For the conventional hearing test, the tested-man has to enter a no-noise chamber and put on earmuffs. The transmission lines of the earmuffs plug into a test apparatus. The sound is generated in the left earmuff or the right earmuff by the operator who is outside the no-noise chamber using the test apparatus. When the tested-man hears that the sound is in the left earmuff or the right earmuff, the tested-man will push the button to show that the sound is in the left earmuff or the right earmuff. The operator will judge that the hearing of the tested-man is normal or ab normal according to the button pushed by the tested-man.

Because the volume of the hearing test apparatus mentioned above is very large, the hearing test apparatus has to be arranged in the hospital or related unit. The tested-man has to go to the hospital or related unit as well. It is inconvenient.

### SUMMARY OF THE INVENTION

In order to solve the above-mentioned problems, an object of the present invention is to provide a portable hearing test apparatus. The volume of the hearing test apparatus is reduced and is arranged in the earphone to be connected to the electronic apparatus. Therefore, it is convenient for the user to bring out the portable hearing test apparatus. Moreover, the portable hearing test apparatus has the anti-noise function, so that the hearing test does not require to test in the no-noise environment.

In order to solve the above-mentioned problems, another object of the present invention is to provide a portable hearing test apparatus with a dual tone multiple frequency signal control module for receiving the audio signal outputted from the portable electronic apparatus. The parameters of the automatic noise elimination module of the portable hearing test apparatus can be set or is adjustable by the dual tone multiple frequency signal control module according to the audio signal outputted from the portable electronic apparatus.

In order to achieve the object of the present invention mentioned above, the portable hearing test apparatus is arranged in an earphone and electrically connected to an electronic apparatus. The electronic apparatus provides an audio signal. The portable hearing test apparatus comprises a microphone, an automatic noise elimination module, a mixer and an output unit. The microphone is arranged inside the earphone. The automatic noise elimination module is arranged inside the earphone and is electrically connected to the microphone. The mixer is arranged inside the earphone and is electrically connected to the automatic noise elimination module and the electronic apparatus. The output unit is arranged inside the earphone and is electrically connected to the mixer. The microphone receives an external noise signal. The automatic noise elimination module converts the external noise signal into an inverse acoustic signal. The automatic noise elimination module sends the inverse acoustic signal to the mixer. An amplitude of the inverse acoustic signal is equal to an amplitude of the external noise signal. A phase of the inverse acoustic signal and a phase of the external noise signal are reverse. The mixer mixes the inverse acoustic signal with the audio signal provided by the electronic apparatus to obtain a mixed signal. The mixer sends the mixed signal to the output unit. The output unit sends out the mixed signal to cancel out the external noise signal, so that only the audio signal provided by the electronic apparatus is heard. A hearing test provided by the portable hearing test apparatus is proceeded with the audio signal.

In an embodiment of the present invention, the audio signal is between 125 Hertz and 8000 Hertz.

In an embodiment of the present invention, the output unit is a loudspeaker.

In an embodiment of the present invention, the electronic apparatus is a portable electronic apparatus.

In an embodiment of the present invention, the portable electronic apparatus is a tablet computer, a notebook computer or a smartphone.

In order to achieve another object of the present invention mentioned above, the portable hearing test apparatus is arranged in an earphone and electrically connected to an electronic apparatus. The electronic apparatus provides an audio signal. The portable hearing test apparatus comprises a microphone, an automatic noise elimination module, a mixer, an output unit and a dual tone multiple frequency signal control module. The microphone is arranged inside the earphone. The automatic noise elimination module is arranged inside the earphone and is electrically connected to the microphone. The mixer is arranged inside the earphone and is electrically connected to the automatic noise elimination module and the electronic apparatus. The output unit is arranged inside the earphone and is electrically connected to the mixer. The dual tone multiple frequency signal control module is arranged inside the earphone and is electrically connected to the electronic apparatus and the automatic noise elimination module. The microphone receives an external noise signal. The automatic noise elimination module converts the external noise signal into an inverse acoustic signal. The automatic noise elimination module sends the inverse acoustic signal to the mixer. An amplitude of the inverse acoustic signal is equal to an amplitude of the external noise signal. A phase of the inverse acoustic signal and a phase of the external noise signal are reverse. The mixer mixes the inverse acoustic signal with the audio signal provided by the electronic apparatus to obtain a mixed signal. The mixer sends the mixed signal to the output unit. The output unit sends out the mixed signal to cancel out the external noise signal, so that only the audio signal provided by the electronic apparatus is heard. A hearing test provided by the portable hearing test apparatus is proceeded with the audio signal. When the hearing test is not proceeded, the dual tone multiple frequency signal control module receives the audio signal provided by the electronic apparatus to set or adjust parameters of the automatic noise elimination module.

In an embodiment of the present invention, the audio signal is between 125 Hertz and 8000 Hertz.

In an embodiment of the present invention, the output unit is a loudspeaker.

In an embodiment of the present invention, the electronic apparatus is a portable electronic apparatus.

In an embodiment of the present invention, the portable electronic apparatus is a tablet computer, a notebook computer or a smartphone.

### BRIEF DESCRIPTION OF DRAWING

FIG. 1 shows a block diagram of the first embodiment of the portable hearing test apparatus of the present invention.

FIG. 2 shows that the portable hearing test apparatus is connected to the electronic apparatus.

FIG. 3 shows a block diagram of the second embodiment of the portable hearing test apparatus of the present invention.

### DETAILED DESCRIPTION OF THE INVENTION

Please refer to following detailed description and figures for the technical content of the present invention.

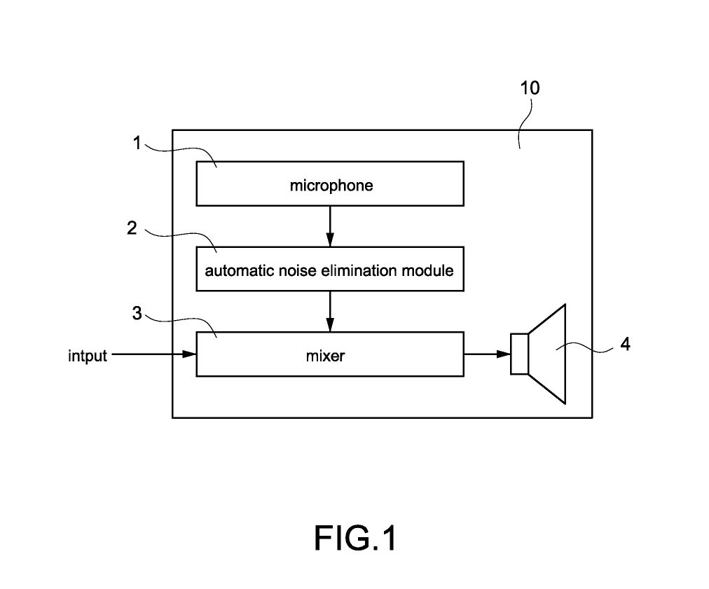

FIG. 1 shows a block diagram of the first embodiment of the portable hearing test apparatus of the present invention. As shown in FIG. 1, the portable hearing test apparatus comprises a microphone 1, an automatic noise elimination module 2, a mixer 3 and an output unit 4.

The microphone 1 is arranged inside the earphone 10. The microphone 1 receives an external noise signal and sends the external noise signal to the automatic noise elimination module 2.

The automatic noise elimination module 2 is arranged inside the earphone 10 and is electrically connected to the microphone 1 to receive the external noise signal outputted from the microphone 1. The automatic noise elimination module 2 converts the external noise signal into an inverse acoustic signal. The automatic noise elimination module 2 sends out the inverse acoustic signal. An amplitude of the inverse acoustic signal is equal to an amplitude of the external noise signal. A phase of the inverse acoustic signal and a phase of the external noise signal are reverse.

The mixer 3 is arranged inside the earphone 10 and is electrically connected to the automatic noise elimination module 2 and an electronic apparatus (not shown in FIG. 1). The mixer 3 receives the inverse acoustic signal outputted from the automatic noise elimination module 2 and an audio signal provided by the electronic apparatus. The audio signal is used for hearing test. The mixer 3 mixes the inverse acoustic signal with the audio signal to obtain a mixed signal. The mixer 3 sends out the mixed signal.

The output unit 4 is arranged inside the earphone 10 and is electrically connected to the mixer 3. The output unit 4 receives the mixed signal and sends out the mixed signal. In FIG. 1, the output unit 4 is a loudspeaker.

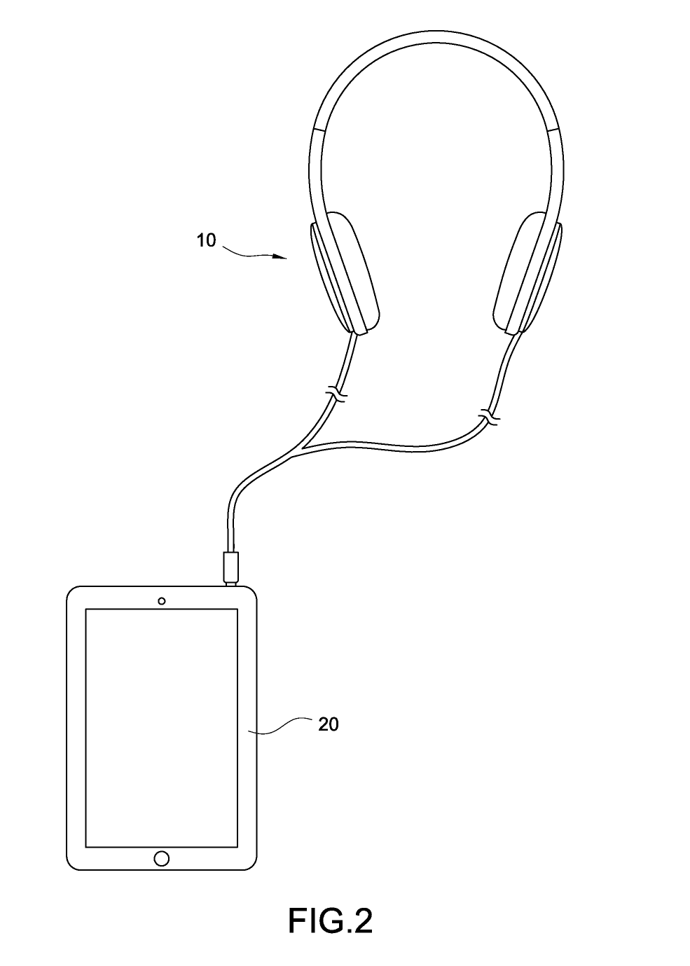

FIG. 2 shows that the portable hearing test apparatus is connected to the electronic apparatus. Please refer to FIG. 1 as well. As shown in FIG. 1 and FIG. 2, when a tested-man is under a hearing test provided by the portable hearing test apparatus, an input side of the earphone 10 plugs into an electronic apparatus 20 (for example, a tablet computer, a notebook computer or a smartphone). The tested-man puts on the earphone 10. The microphone 1 receives the external noise signal and sends the external noise signal to the automatic noise elimination module 2. The automatic noise elimination module 2 converts the external noise signal into the inverse acoustic signal. The amplitude of the inverse acoustic signal is equal to the amplitude of the external noise signal. The phase of the inverse acoustic signal and the phase of the external noise signal are reverse. The automatic noise elimination module 2 sends the inverse acoustic signal to the mixer 3. The electronic apparatus 20 outputs the audio signal which is between 125 Hertz and 8000 Hertz to the mixer 3. The mixer 3 mixes the inverse acoustic signal with the audio signal to obtain the mixed signal. The mixer 3 sends the mixed signal to the output unit 4. The output unit 4 sends out the mixed signal (comprising the inverse acoustic signal) to cancel out the external noise signal, so that the tested-man does not hear the external noise signal and only hears the audio signal which is between 125 Hertz and 8000 Hertz and is provided by the electronic apparatus 20. The hearing test provided by the portable hearing test apparatus is proceeded with the audio signal.

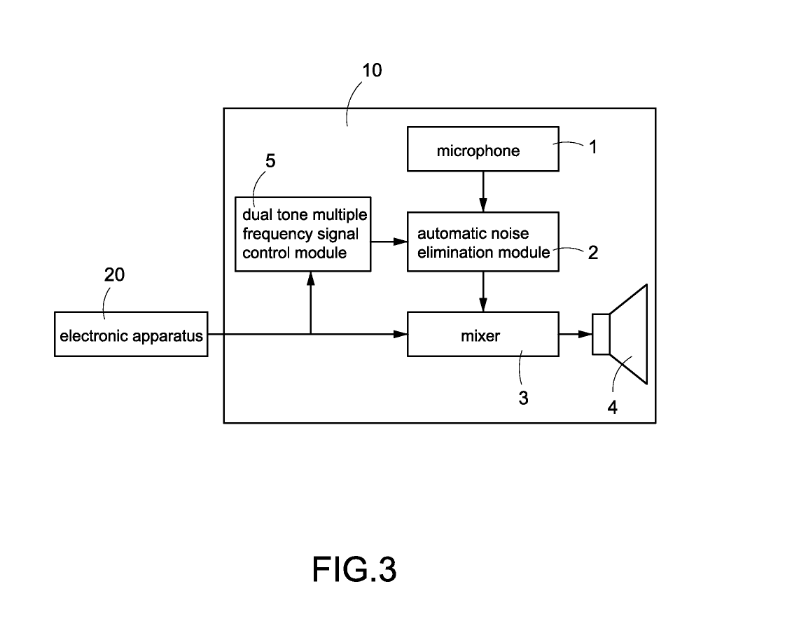

FIG. 3 shows a block diagram of the second embodiment of the portable hearing test apparatus of the present invention. As shown in FIG. 3, a dual tone multiple frequency signal control module 5 (namely, the dialing tones generated by the conventional telephone dialing) is arranged between the electronic apparatus 20 and the automatic noise elimination module 2. The dual tone multiple frequency signal control module 5 is used to set or adjust parameters of the automatic noise elimination module 2. The dual tone multiple frequency signal control module 5 properly converts the audio signal provided by the electronic apparatus 20 into signals for controlling the automatic noise elimination module 2. For example, if the value of the control position “12” of the automatic noise elimination module 2 would like to be set as “34”, then the electronic apparatus 20 has to send out “77#1234”, wherein the “*77#” is the beginning identification number which can be changed to other number, but it works only if it complies. When the amplitude or the phase for converting signals of the portable hearing test apparatus are distorted, if the hearing test is not proceeded, the dual tone multiple frequency signal control module 5 sets or adjusts the parameters of the automatic noise elimination module 2. Therefore, no extra I/O apparatus is required for setting or adjusting the parameters of the automatic noise elimination module 2.

Moreover, after the hearing test is finished, the tested-man or the operator can use a peripheral apparatus (such as a printer) to print out the hearing test result through the wireless transmission module (such as Bluetooth module or wifi module) of the electronic apparatus 20 or through the Internet.

Although the present invention has been described with reference to the preferred embodiment thereof, it will be understood that the invention is not limited to the details thereof Various substitutions and modifications have been suggested in the foregoing description, and others will occur to those of ordinary skill in the art. Therefore, all such substitutions and modifications are intended to be embraced within the scope of the invention as defined in the appended claims.

What is claimed is:

1. A portable hearing test apparatus arranged in an earphone and electrically connected to an electronic apparatus, the electronic apparatus providing an audio signal, the portable hearing test apparatus comprising:
a microphone arranged inside the earphone;
an automatic noise elimination module arranged inside the earphone and electrically connected to the microphone;
a mixer arranged inside the earphone and electrically connected to the automatic noise elimination module and the electronic apparatus; and
an output unit arranged inside the earphone and electrically connected to the mixer,
wherein the microphone receives an external noise signal; the automatic noise elimination module converts the external noise signal into an inverse acoustic signal; the automatic noise elimination module sends the inverse acoustic signal to the mixer; an amplitude of the inverse acoustic signal is equal to an amplitude of the external noise signal; a phase of the inverse acoustic signal and a phase of the external noise signal are reverse; the mixer mixes the inverse acoustic signal with the audio signal provided by the electronic apparatus to obtain a mixed signal; the mixer sends the mixed signal to the output unit; the output unit sends out the mixed signal to cancel out the external noise signal, so that only the audio signal provided by the electronic apparatus is heard; a hearing test provided by the portable hearing test apparatus is proceeded with the audio signal.

2. The portable hearing test apparatus in claim 1, wherein the audio signal is between 125 hertz and 8000 hertz.

3. The portable hearing test apparatus in claim 1, wherein the output unit is a loudspeaker.

4. The portable hearing test apparatus in claim 1, wherein the electronic apparatus is a portable electronic apparatus.

5. The portable hearing test apparatus in claim 4, wherein the portable electronic apparatus is a tablet computer, a notebook computer or a smartphone.

6. A portable hearing test apparatus arranged in an earphone and electrically connected to an electronic apparatus, the electronic apparatus providing an audio signal, the portable hearing test apparatus comprising:
a microphone arranged inside the earphone;
an automatic noise elimination module arranged inside the earphone and electrically connected to the microphone;
a mixer arranged inside the earphone and electrically connected to the automatic noise elimination module and the electronic apparatus;
an output unit arranged inside the earphone and electrically connected to the mixer; and
a dual tone multiple frequency signal control module arranged inside the earphone and electrically connected to the electronic apparatus and the automatic noise elimination module,
wherein the microphone receives an external noise signal; the automatic noise elimination module converts the external noise signal into an inverse acoustic signal; the automatic noise elimination module sends the inverse acoustic signal to the mixer; an amplitude of the inverse acoustic signal is equal to an amplitude of the external noise signal; a phase of the inverse acoustic signal and a phase of the external noise signal are reverse; the mixer mixes the inverse acoustic signal with the audio signal provided by the electronic apparatus to obtain a mixed signal; the mixer sends the mixed signal to the output unit; the output unit sends out the mixed signal to cancel out the external noise signal, so that only the audio signal provided by the electronic apparatus is heard; a hearing test provided by the portable hearing test apparatus is proceeded with the audio signal; when the hearing test is not proceeded, the dual tone multiple frequency signal control module receives the audio signal provided by the electronic apparatus to set or adjust parameters of the automatic noise elimination module.

7. The portable hearing test apparatus in claim 6, wherein the audio signal is between 125 hertz and 8000 hertz.

8. The portable hearing test apparatus in claim 6, wherein the output unit is a loudspeaker.

9. The portable hearing test apparatus in claim 6, wherein the electronic apparatus is a portable electronic apparatus.

10. The portable hearing test apparatus in claim 9, wherein the portable electronic apparatus is a tablet computer, a notebook computer or a smartphone.
